(19) United States
(12) Patent Application Publication
(10) Pub. No.: US 2006/0247714 A1
(43) Pub. Date: Nov. 2, 2006

(54) Glass-to-metal feedthrough seals having improved durability particularly under AC or DC bias

(76) Inventors: William J. Taylor, Anoka, MN (US); Zhi Fang, Maple Grove, MN (US); William D. Wolf, St. Louis Park, MN (US); Shawn D. Knowles, St. Francis, MN (US)

Correspondence Address: MEDTRONIC, INC., 710 MEDTRONIC PARK, MINNEAPOLIS, MN, 55432-9924, US

(21) Appl. No.: 11/116,968

(22) Filed: Apr. 28, 2005

(51) Int. Cl. A61N 1/375

(52) U.S. Cl. 607/36

(57) ABSTRACT

A hermetic implantable medical device (IMD) is provided with a single or multi-pin arrangement including selected glass to metal seals for a feedthrough including a ceramic disk member coupled to the sealing glass surface in potential contact with body fluids. By judicious selection of component materials (ferrule, seal insulator and pin) provides for either compression or match seals for electrical feedthroughs (having a single or multi-pin array) provide corrosion resistance and biocompatibility required in IMDs. The resultant feedthrough configuration accommodates one pin within a single ferrule or at least two pins in a single ferrule having a pin surrounded by insulator material (e.g., alumina ceramic, zirconia ceramic, zirconia silicate ceramic, mullite, each having higher melting points than the sealing glass distributed around the pin within the ferrule, or feldspar porcelain materials or alumino-silicate glasses having a lower melting point than the sealing glass) distributed around the pin within the ferrule.

### FIELD OF THE INVENTION

This invention relates to electrical feedthrough devices and particularly to single and multiple pin electrical feedthrough assemblies for providing electrical communication between electrical components such as medical electrical leads and diverse sensors and operative circuitry housed within the interior of a hermetically sealed implantable medical device (IMD).

### BACKGROUND OF THE INVENTION

There are numerous applications where it is necessary to penetrate a sealed container with a plurality of electrical leads so as to provide electrical access to and from electrical components enclosed within. One such application for which the present invention has particular but not limited utility is in body implantable pulse generators (e.g. for treatment of bradycardia, tachyarrhythmia or for muscle or nerve stimulation) which includes neurostimulation devices, deep brain stimulators, and the like, herein referred to as implantable pulse generators (IPG's). The heart pacemaker is a well-known example of one type of IPG. Typical devices of this type are formed of a metal container housing the electrical and power source components of the IPG with a lid or the like welded to the container to close the device and provide it with a hermetic seal. An electrical lead is electrically connected to the IPG by means of attachment to one or more feedthroughs which penetrate the container but maintain the hermetically sealed environment thereof. A typical feedthrough consists of an external metal part (a frame or ferrule) into which preformed solid or sintered glass part is sealed. Within the glass part, one or more metal leads (pins) are sealed. Since the reliability of critical implantable medical devices depend on hermetic sealing of various components, the integrity of the glass to metal seals used between the internal electrical components and the human body is of paramount importance.

In many implantable medical devices, metals which have long-term corrosion resistance and biocompatibility are needed to provide years of reliable service since maintenance or repair possibilities for the devices are extremely limited. Moreover, since such devices are sometimes lifesaving for the patient, failures of the feedthrough materials can have catastrophic consequences. Therefore, metals like titanium, niobium, tantalum, platinum and the like are use due to their well-known superior corrosion resistance and biocompatibility.

Other types of implantable medical devices that require hermetic couplings to operative circuitry disposed within a housing include implantable cardioverter-defibrillators (ICDs), drug pumps, and the like. Herein all such devices, including IPGs, medical electrical leads and associated sensors are referred to from time to time herein by the phrase implantable medical devices (IMDs).

As IMDs have undergone development, they have become smaller yet more electronically sophisticated, making it necessary to include more and more functions into smaller and smaller containers. This translates into a need for multi-pin feedthroughs carried by small, usually slim, containers. Multi-pin arrangements of feedthrough pins for diverse IMDs have been suggested before. For example, in U.S. Pat. No. 4,874,910 issued to McCoy, a number of flat pins are shown traversing a hermetic glass seal in a linear array. Or, in Neilsen et al, “Development of Hermetic Micro miniature Connections”, Journal of Elastomeric Packaging. December 1991, Vol 113/405-409, the stresses on a compression seal for a multi-pin device are modeled. However, the successful combination of materials which include the corrosion resistance and biocompatibility required for an implantable medical device have not been disclosed.

In addition, Applicants hereby incorporate by reference U.S. Pat. No. 4,315,974 to Athearn et al. entitled, “Electrochemical Cell with Protected Electrical Feedthrough,” which issued 16 Feb. 1982. Among other things, the '974 patent purports to propose use of a protective inner ceramic body which is sealed to an inner glass portion of a seal means in a protective relationship so as to shield exposed inner portions of the glass from inner attack by incompatible contents of the device. The '974 patent notes that not all of the inner glass need be shielded, only those portions exposed to incompatible components. The invention contemplates complete inner shielding as well as partial inner shielding of the glass surface. Notably however, the '974 patent does not purport to deal with corrosion protection in the presence of applied bias voltages and/or currents nor with the aspect of direct or indirect interaction with bodily fluids. Such corrosion can reduce the expected service life of many IMDs. Applicants hereby incorporate U.S. Pat. No. 6,090,503 entitled, “Body Implanted Device with Electrical Feedthrough,” which issued 18 Jun. 2000 the contents of which are also hereby incorporated by reference herein.

Thus, a need in the art exists for a family of robust bias-tolerant feedthrough assemblies that provide corrosion protection for diverse IMDs especially in the presence of applied bias voltages and/or currents as well as protection in the direct or indirect presence of diverse bodily fluids.

### SUMMARY

This invention, by judicious selection and combination of component materials (ferrule, seal insulator and pin) provides for either compression or match seals for electrical feedthroughs, the pins of which are arranged either singularly or in a multi-pin array together with corrosion resistance and biocompatibility needed in an IMD. The resultant feedthrough configuration accommodates a single pin arranged within a single ferrule or multiple pins with in a single ferrule wherein each pin is surrounded by one or more insulator materials (e.g., alumina ceramic, fused silica, sapphire, ruby, zirconia ceramic, zirconia silicate ceramic, mullite, each having a higher melting point than the sealing glass distributed around the pin with in the ferrule, or feldspar porcelain materials or alumino-silicate glasses each having a lower melting point than the sealing glass distributed around the pin within the ferrule). The number and configuration of the pins can be modified beyond at least two arranged pins (e.g., expanded in number: along an axis, in pairs, offset, linearly etc.) to any desired number, pattern or configuration. A linear configuration results in easy identification of the pins and facilitates automated connection therewith and maintains device slimness even when a large number of pins are included in the feedthrough arrangement. Arranging the pins into a consistent pattern or other arrangement provides easy access allowing the use of a plug-in electrical connector to facilitate rapid manual or automated processes to connect multiple termination electrical connections to IMD circuitry and related components.

A pair of ceramic disks coupled to opposing distal portions of each conductive pin can provide superior corrosion resistance to the feedthrough pin and related components. Alternatively, a single disk disposed on the side of a pin that might be expected to encounter, either directly or indirectly, various body fluids can also be practiced according to the invention. As noted above, these insulator materials can be fabricated from alumina ceramic, fused silica, sapphire, ruby, zirconia ceramic, zirconia silicate ceramic, mullite, each having a higher melting point than the sealing glass distributed around the pin within the ferrule, or feldspar porcelain materials or alumino-silicate glasses each having a lower melting point than the sealing glass distributed around the pin within the ferrule. The feedthrough assemblies according to the present invention represent a hermeticity and reliability improvement relative to gold-braze ceramic-to-metal seals especially under DC or AC bias (e.g., a low magnitude direct current bias used for example in conjunction with certain implantable sensors or the like). The inventors hereof cross reference U.S. Pat. No. 5,817,984, U.S. Pat. 5,866,851, U.S. Pat. No. 5,821,011 and incorporate the contents as if fully set forth herein (with the noticeable exception of the Au-braze FT designs described and depicted in the '851 and '984 patents). On advantage of the present invention involves the use of a ceramic material (e.g., a disk) bonded to the surface of a glass-to-metal seal to improve the impedance performance of the glass-to-metal feedthrough in body-implantable applications.

The inventors hereof emphasize that the improvement in DC bias resistance of glass-to-metal seals relative to the traditional ceramic-to-metal seals for applications involving direct and for indirect body fluid contact. Use of generic terms such as “implantable” could imply power sources or capacitors, which deliver direct current (DC) signals via glass-to-metal seals. These components while technically “implantable,” do not typically come into contact with body fluids, as they are enclosed within a pacing or other active implantable medical device (IMD).

Thus, by illustration and without limitation the present invention provides several advantages in producing robust feedthrough assemblies that might be subjected to bias voltage and/or electrical current while chronically subject to bodily fluids and related substances, other advantages will become clear to those of skill in the art upon review of the present patent document, including:
1. A glass-to-metal seal for direct contact with body fluids that exhibits improved glass durability;
2. A glass-to-metal seal for indirect contact with body fluids that exhibits improved glass durability;
3. A glass-to-metal seal for conveying a continuous DC or alternating current (AC) signal that exhibits improved DC- or AC-bias performance and glass robustness;
4. A glass-to-metal seal for conveying a modulated DC or AC signal that exhibits improved DC or AC bias and glass robustness;
5. A glass-to-metal seal containing a glass having free-flowing properties at sealing temperatures and that readily makes contact with and bonds to electrical conductor materials (without the aid of forming weights or other compression techniques);
6. A glass-to-metal seal containing a glass that does not free-flow under its own load at sealing temperatures and thereby requires forming weights (or other compression techniques) to induce sealing contact to conductor materials;
7. A glass-to-metal seal containing a ceramic structure bonded to an adjacent glass surface wherein said ceramic structure covers a substantial portion of the glass surface which surface has the potential to make sustained contact with body fluids; and
8. A glass-to-metal seal not containing a ceramic disc bonded to the glass surface and including a free flowing glass exhibiting improved glass durability and/or glass with more durable exterior layer (e.g., an improved functional gradient).

The foregoing and other aspects and features of the present invention will be more readily understood from the following detailed description of the embodiments thereof, when considered in conjunction with the drawings, in which like reference numerals indicate similar structures throughout the several views.

### BRIEF DESCRIPTION OF THE DRAWINGS

FIG. 1 is cutaway perspective view of an exemplary IPG.

FIG. 2 rows a cross-section taken along line 2-2 in FIG. 1 of the IPG interior and feedthrough.

FIGS. 3 and 4 show a cross-sectional and elevational views respectively of a first configuration according to the invention (separate insulator for each pin).

FIGS. 5 and 6 show a cross-sectional and elevational views respectively of a second configuration according to the invention (common insulator).

FIGS. 7 and 8 show similar views respectively of an optional ceramic disc embodiment.

FIGS. 9A and 9B depict in cross-section a so-called uni-polar feedthrough having a single conductive pin surrounded by a sealing glass and surrounded by the periphery of an aperture formed in a metallic housing of a device, and a similar feedthrough having a sleeve according to various embodiments of the invention.

FIGS. 10 and 11 are tables providing a matrix of material combinations to produce a variety of robust feedthrough assemblies according to the present invention.

### DETAILED DESCRIPTION OF THE ILLUSTRATED EMBODIMENTS

While this invention may be embodied in many different forms, there are shown in the drawings and described in detail herein specific preferred embodiments as applied to IPG's. The present invention is exemplified as to its principles and is not meant to be limited to the particular embodiments illustrated.

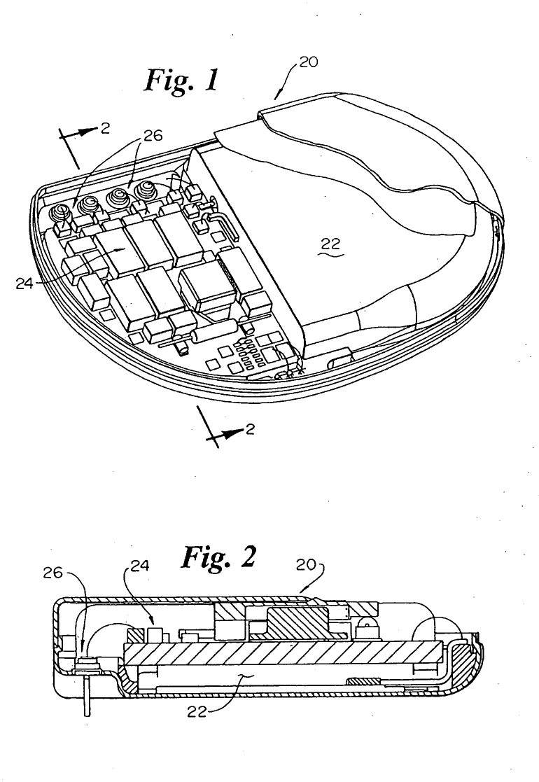

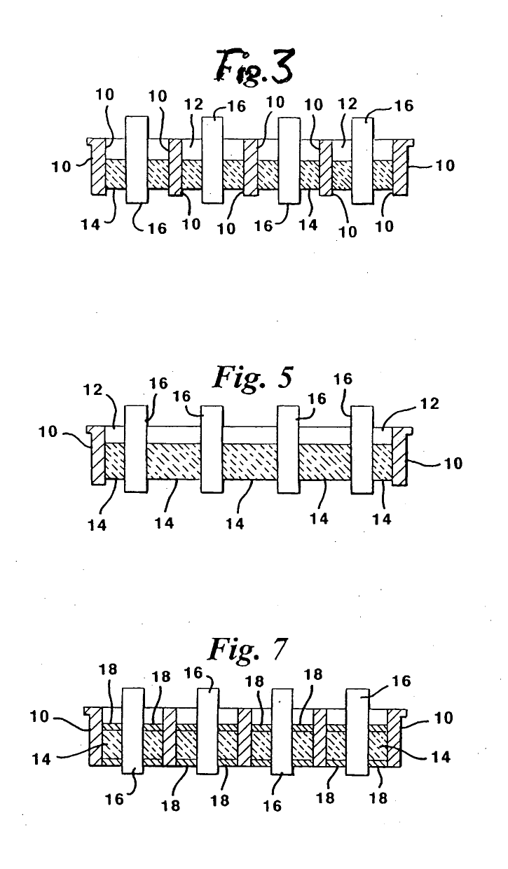

Referring first to FIGS. 1 and 2, an IPG 20 is shown generically. It includes a battery section 22, a circuit section 24 and a linearly arranged plurality of feedthroughs 26.

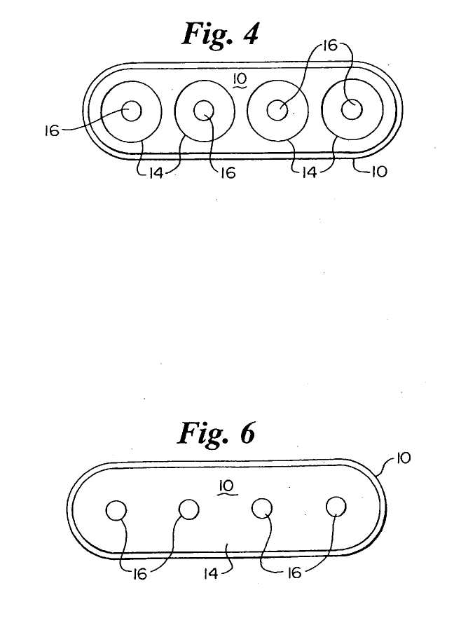

Different feedthrough configurations may be used in the device illustrated in FIGS. 1 and 2 according to this invention and welded into place as a unit in an aperture of the IPG 20. Configurations are shown in FIGS. 3-4 and 5-6. A first linear configuration is shown in FIGS. 3 and 4 having an elongated titanium ferrule 10 having a plurality of openings 12 extending there through. The ferrule 10 can be provided by conventional machining, stamping or chemical etching operations, etc. Each of the openings 12 receives a linear array of discrete sealing insulator bodies 14 more specifically described hereinbelow as to choice of materials and which in turn carry a linear array of pins 16 (more specifically disclosed herein below as to choice of materials) which are preferably centered in each of the openings 12.

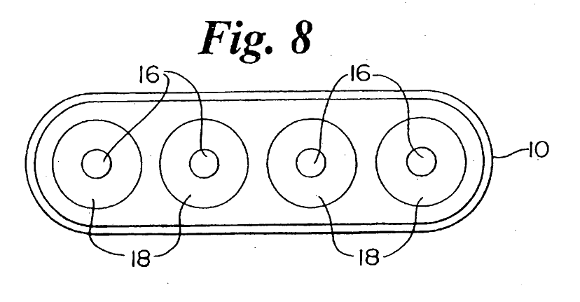

Another linear configuration is shown in FIGS. 5 and 6, also having an elongated titanium ferrule 10 having a single elongated opening 12 there through which receives a single elongate sealing body 14 (more specifically described herein below as to materials) and which in turn carries a linear array of pins 16 centered in the opening 12.

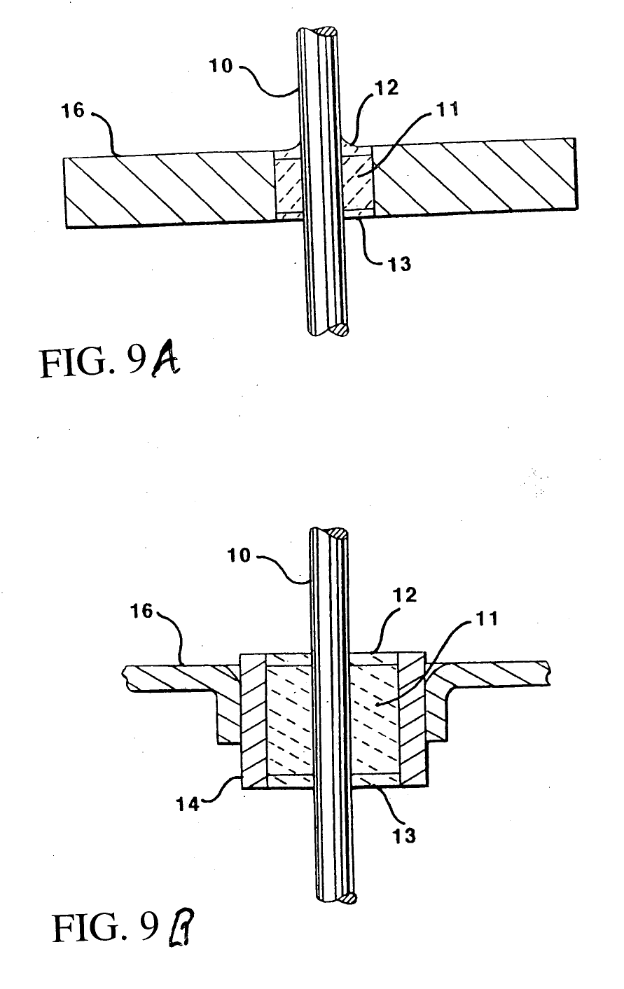

FIGS. 7 and 8 show an embodiment similar to FIGS. 3 and 4 optionally including an array of discrete upper and/or lower ceramic disks (optional) 18 covering the insulators bodies 12 and surrounding pins 16. A similar option (not shown) may be included in the configuration of FIGS. 5 and 6 wherein a simple elongated ceramic disc is included on the upper and/or lower surfaces of the insulator body 14.

Two ceramic bodies similar to the arrangement shown in FIG. 7 may be used to provide electrical insulation with glass in between. Not all glasses deform easily at their sealing temperatures. High viscosity glasses may require mechanical deformation by weights from above. Often this “weight system” requires direct contact with the sealing glass by a non-adherent material such as graphite. However, as was stated earlier, with specific glass compositions required when sealing glass to titanium, graphite may not be as non-adherent as desired. Therefore, mechanical deformation of the sealing glass may require providing a “sandwich” with the glass located between the electrically non-conductive material which do not adhere to the graphite but adhere to the glass when sealing occurs.

The ceramic body or bodies provide several advantages; namely, they provide excellent prophylactic function vis-a-vis corrosion of the insulating material (e.g., glass) 14 and the ferrule or periphery of the surrounding metallic substrate 10 particularly when coupled to opposing sides of a feedthrough assembly according to the invention. In addition, for feedthrough assemblies that subjected to prolonged exposure to AC or DC bias voltage and/or current and in the direct or indirect contact with body fluids a ceramic disk provides even greater protection thereby extending the expected service life of the component 20. As shown during extensive testing by the inventors hereof, the foregoing properties of the inventive feedthrough assembly are even more impressive when the feedthrough assembly is subjected directly or indirectly to body fluids.

In accordance with this invention a single pin or multi-pin arrangement is carried out by the joining methods and material combinations. In one embodiment, a feedthrough utilizes glass-to-metal seals. Glass-to-metal seals incorporate an outer ring or ferrule 10 comprised of a weldable grade of titanium or titanium-containing alloy as shown in FIGS. 3-8. The insulator 14 is comprised of a boro-alumino (1), boro-alumino silicate (2) or boro silicate (3) glass with a wide range of thermal expansions to match biostable pin materials such as tantalum, niobium, niobium-titanium alloy, platinum, platinum alloys, titanium and alloys of titanium.

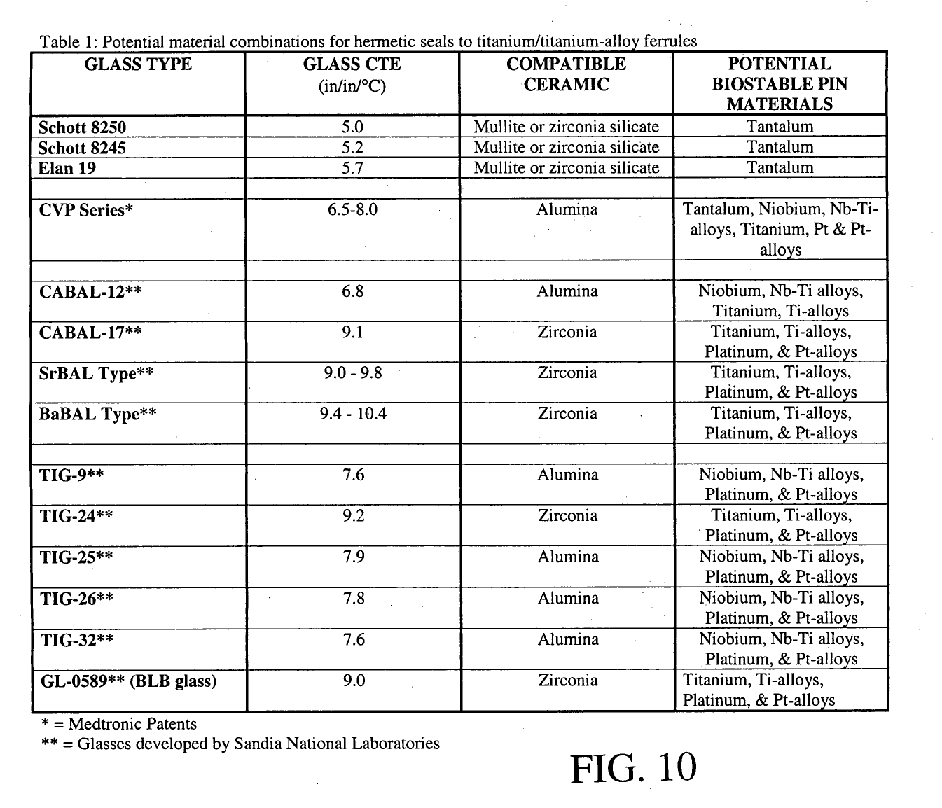

FIGS. 9A and 9B depict in cross-section two embodiments of a so-called uni-polar feedthrough assembly each having a single conductive pin surrounded by a sealing glass and surrounded by the periphery of an aperture formed in a metallic housing of a device, and a similar feedthrough having a sleeve according to various embodiments of the invention. Referring to FIG. 9A a portion of a body-implanted device with an electrical feedthrough is shown. The feedthrough includes a center pin or terminal 10, a glass seal member 11, and top and bottom end caps 12 and 13 respectively. In the arrangement of FIG. 1, the feedthrough is positioned such that top end cap 12 and bottom end cap 13 and glass seal member 11 extend through an opening in container 16. This arrangement and that of FIG. 2 wherein the feedthrough includes a sleeve or header 14 are typical feedthrough seal arrangements that may make use of the invention. Other arrangements may be used as well and may take any configuration in which the metal is wetted by the glass. Referring now to FIG. 9B, another embodiment of the invention is illustrated. The feedthrough includes a terminal 10 extending through a glass seal 11 bounded by top end cap 12, bottom end cap 13 and sleeve or header 14. In practice each body-implanted device may have multiple feedthroughs. Sleeve 14 may be welded into an opening in the housing of the body-implanted device such as container 16 of, for example, titanium or titanium alloy. During assembly, the feedthrough is placed in an oven or furnace and heated causing the glass seal member to wet the metallic components to form a hermetic seal between the glass and the metal components.

Since electrical feedthroughs used in body-implanted devices may inadvertently come into contact with body fluids, it is desirable that terminal 10 be made of a bio-stable material. For example, terminal 10 may consist of niobium, titanium, tantalum, platinum or a platinum-iridium alloy. However, the use of niobium, or tantalum or alloys thereof may be inappropriate because of their susceptibility to hydrogen embrittlement. This is especially true in direct current feedthroughs at the negative terminal where hydrogen embrittlement can occur as a result of the exposure of the terminal to body fluids. In such situations it is preferable to use platinum, platinum-iridium alloys, pure titanium or titanium metallurgically clad to a thickness of about 50 to 300 microinches over tantalum or niobium because they are less susceptible to hydrogen embrittlement. The particular material chosen is based upon its compatibility with the thermal expansion characteristics of the glass seal.

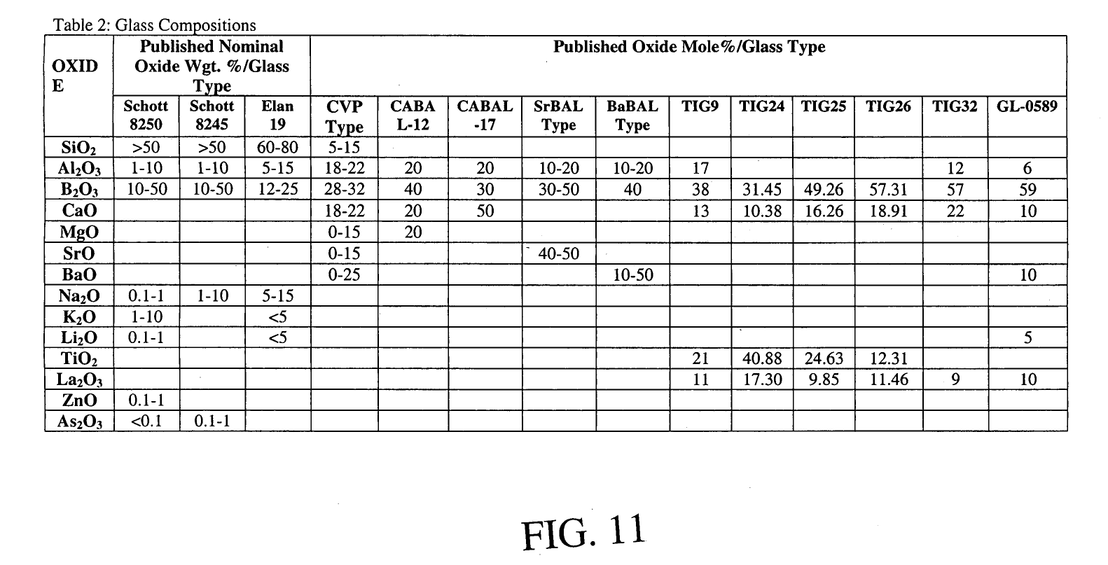

Specific combinations of materials usable according to the invention are shown in the Tables appended hereto as FIG. 10 and FIG. 11. FIGS. 10 and 11 are tables providing a matrix of material combinations to produce a variety of robust feedthrough assemblies according to the present invention. The combinations provide robust performance under a variety conditions; however, during conditions involving AC or DC bias at the pin that also involve direct or indirect exposure to body fluids, the inventive combination provides increased utility over the prior art.

Of the foregoing material combinations in single or linear array, glass types (1), (2) and (3) and the ceramic type provide reliable seals.

This completes the description of the preferred and alternate embodiments of the invention. Those skilled in the art may recognize other equivalents to the specific embodiments described herein which equivalents are intended to be encompassed by the claims hereto.

It should be understood that, certain of the above-described structures, functions and operations of the pacing systems of the illustrated embodiments are not necessary to practice the present invention and are included in the description simply for completeness of an exemplary embodiment or embodiments. It will also be understood that there may be other structures, functions and operations ancillary to the typical operation of an implantable pulse generator that are not disclosed and are not necessary to the practice of the present invention.

1. In an implantable medical device (IMD) comprising a hermetically sealed case and a feedthrough hermetically sealed in an aperture of the case, wherein the feedthrough assembly endures prolonged exposure to at least one of body fluids and continuous or modulated AC or DC bias, said improvement comprising:
a feedthrough comprising a ferrule of biocompatible, corrosion resistant metal and having an aperture disposed there through;
an insulator body sealed to the ferrule within the aperture of the ferrule;
a conductive pin extending through the aperture of the ferrule in sealing engagement with the insulator body; and
a substantially planar disk coupled to opposing exposed portions of the insulator body and formed of a compatible ceramic material comprising one of the group: mullite, zirconia silicate, alumina, zirconia;
wherein the biocompatible, corrosion resistant metal of the ferrule is selected from the group consisting of titanium, titanium alloys, niobium/titanium alloys and wherein the insulator body comprises a glass having a nominal coefficient of thermal expansion of approximately between 5.0 and 10.4.

2. An IMD according to claim 1, wherein a conductive pin is disposed in a corresponding aperture.

3. An IMD according to claim 1, wherein the conductive pin comprises at least two pins arranged in a linear array and each pin is disposed in a corresponding aperture.

4. An IMD according to claim 1, wherein the IMD comprises an implantable cardioverter-defibrillator.

5. An IMD according to claim 1, wherein the IMD comprises an implantable cardiac pacemaker.

6. An IMD according to claim 1, wherein the IMD comprises an implantable deep brain stimulation device.

7. An IMD according to claim 1, wherein the IMD comprises a neurological stimulator.

8. An IMD according to claim 1, wherein the IMD comprises an implantable drug delivery pump.

9. An IMD according to claim 1, wherein the IMD comprises an implantable pressure sensor.

10. An IMD according to claim 1, wherein the insulator body comprises a single common body surrounding all of the pins.

11. An IMD according to claim 1, wherein an individual insulator body surrounds individual pin.

12. An IMD according to claim 1, wherein the substantially planar disk couples to only a portion of the periphery of one of the pin and the ferrule.

13. A method of fabricating feedthrough assembly for an implantable medical device (IMD) which includes a hermetically sealed case and an improved feedthrough hermetically sealed in an aperture of the case, wherein the feedthrough assembly endures prolonged exposure to at least one of body fluids and AC or DC bias, said improvement comprising:
providing a ferrule of a biocompatible, corrosion resistant metal and having an aperture disposed there through;
sealing an insulator body to the ferrule within the aperture of the ferrule; and
inserting a conductive pin through the aperture of the ferrule in sealing engagement with the insulator body; and
placing a substantially planar disk to the portion of the insulator body in potential contact with body fluids wherein the disk comprises a compatible ceramic material from the group: mullite, zirconia silicate, alumina, and zirconia;
wherein the biocompatible, corrosion resistant metal of the ferrule is selected from the group consisting of titanium, titanium alloys, niobium/titanium alloys and
wherein the insulator body comprises a glass having a nominal coefficient of thermal expansion of approximately between 5.0 and 10.4.

14. A method according to claim 13, wherein a conductive pin is disposed in a corresponding aperture.

15. A method a cording to claim 13, wherein the conductive pin comprises at least two pins arranged in a linear array and each pin is disposed in a corresponding aperture.

16. A method according to claim 13, wherein the IMD comprises an implantable cardioverter-defibrillator.

17. A method according to claim 13, wherein the IMD comprises an implantable cardiac pacemaker.

18. A method according to claim 13, wherein the IMD comprises an implantable deep brain stimulation device.

19. A method according to claim 13, wherein the IMD comprises a neurological stimulator.

20. A method according to claim 13, wherein the IMD comprises an implantable pressure sensor.
